(19) United States
(12) Patent Application Publication
(10) Pub. No.: US 2023/0039086 A1
(43) Pub. Date: Feb. 9, 2023

(54) BMS-986165 CRYSTAL FORM, PREPARATION METHOD THEREFOR AND USE THEREOF

(71) Applicant: CRYSTAL PHARMACEUTICAL (SUZHOU) CO., LTD., Jiangsu (CN)

(72) Inventors: Minhua CHEN, Jiangsu (CN); Hongyan ZHU, Jiangsu (CN)

(21) Appl. No.: 17/785,496

(22) PCT Filed: Dec. 15, 2020
(86) PCT No.: PCT/CN2020/136589

§ 371 Date: Jun. 15, 2022

(30) Foreign Application Priority Data
Dec. 17, 2019 (CN) 201911379338.1
Dec. 17, 2019 (CN) 201911382328.3

(51) Int. Cl. C07D 403/12

(52) U.S. Cl. CPC C07D 403/12; C07B 2200/13

(57) ABSTRACT

A crystal form of a compound I and a preparation method therefor, a pharmaceutical composition containing the crystal form, and a use of the crystal form in the preparation of a TYK2 inhibitor drug and a drug for treating psoriasis, systemic lupus erythematosus, and Crohn's disease. The crystallization of the compound I has one or more improved properties compared to the existing technology, and has an important value to the future optimization and development of the drug.

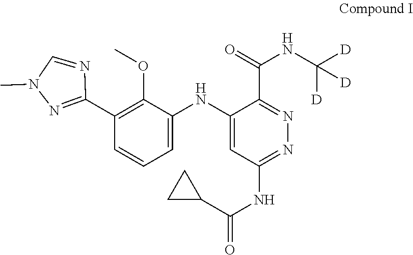 [SMILES: [2H]C([2H])([2H])NC(=O)c1nnc(NC(=O)C2CC2)cc1Nc1cccc(-c2ncn(C)n2)c1OC]

### TECHNICAL FIELD

The present disclosure pertains to the field of chemical crystallography, particularly relates to crystalline forms of BMS-986165, processes for preparation and uses thereof.

### BACKGROUND

Tyrosine kinase 2 (TYK2) is an intracellular signaling kinase that mediates signaling of interleukin-23 (IL-23), interleukin-12 (IL-12) and Type I interferon (IFN), which are cytokines involved in inflammatory and immune responses.

BMS-986165 is the first and only novel, oral, selective TYK2 inhibitor which is used for the treatment of multiple immune-mediated diseases such as psoriasis, psoriatic arthritis, lupus and inflammatory bowel disease. The phase III clinical study results announced in November 2020 reveal that BMS-986165 shows positive clinical effects in the treatment of moderate to severe plaque psoriasis. In addition, BMS-986165 also shows good therapeutic effects in treatment of systemic lupus erythematosus and Crohn's disease.

The chemical name of BMS-986165 is 6-(cyclopropanecarboxamido)-4-((2-methoxy-3-(1-methyl-1H-1,2,4-triazol-3-yl)phenyl)amino)-N-(methyl-d3)pyridazine-3-carboxamide (hereinafter referred to as “Compound I”), and the structure is shown as follows:

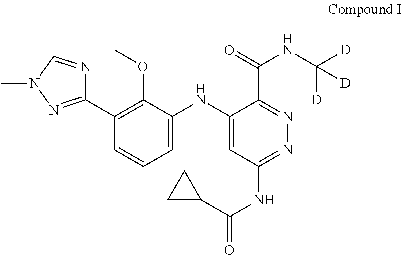 [SMILES: [2H]C([2H])([2H])NC(=O)c1nnc(NC(=O)C2CC2)cc1Nc1cccc(-c2ncn(C)n2)c1OC]

A crystalline form is a solid material whose constituents are arranged in a highly ordered microscopic structure, forming a crystal lattice that extends in all directions. Polymorphism refers to the phenomenon that a compound exists in two or more than two crystalline forms. Different crystalline forms have different physicochemical properties and can affect drug's in vivo dissolution and absorption, which further affect drug's clinical efficacy and safety to some extent. In particular, for poorly soluble drugs, the effects of crystalline forms will be greater. Therefore, drug polymorphism is an important part of drug research and drug quality control.

WO2018183656A1 disclosed crystalline form A of Compound I (hereinafter referred to as Form A) and process for preparation. Form A disclosed in WO2018183656A1 is the only known crystalline form of Compound I free form. The inventors of the present disclosure repeated the preparation method disclosed in WO2018183656A1 to obtain Form A and conducted characterizations. The results show that Form A has poor compressibility and high adhesiveness. Therefore, it is still necessary to develop a crystalline form of Compound I, which has good stability, good compressibility and low adhesiveness for the development of drugs containing Compound I.

The inventors of the present disclosure put a lot of creative work, and then surprisingly discovered crystalline forms CSI and CSII of Compound I provided by the present disclosure, which has advantages in physiochemical properties, formulation processability, bioavailability, etc. For example, crystalline forms CSI and CSII of Compound I has advantages in at least one aspect of melting point, solubility, hygroscopicity, purification ability, stability, adhesiveness, compressibility, flowability, in vitro and in vivo dissolution, and bioavailability, etc. In particular, crystalline forms CSI and CSII of Compound I have good physicochemical stability, good mechanical stability, good compressibility and low adhesiveness, which solve the problems existing in prior arts and are of great significance for the development of drugs containing Compound I.

### SUMMARY

The main objective of the present disclosure is to provide novel crystalline forms of Compound I, preparation method and use thereof.

According to the objective of the present disclosure, crystalline form CSI of Compound I is provided (hereinafter referred to as “Form CSI”).

In one aspect provided herein, the X-ray powder diffraction pattern of Form CSI comprises characteristic peaks at 2theta values of 3.2°±0.2°, 5.6°±0.2° and 8.6°±0.2° using CuKα radiation.

Furthermore, the X-ray powder diffraction pattern of Form CSI comprises one or two or three characteristic peaks at 2theta values of 11.8°±0.2°, 14.2°±0.2° and 15.0°±0.2° using CuKα radiation. Preferably, the X-ray powder diffraction pattern of Form CSI comprises three characteristic peaks at 2theta values of 11.8°±0.2°, 14.2°±0.2° and 15.0°±0.2° using CuKα radiation.

Furthermore, the X-ray powder diffraction pattern of Form CSI comprises one or two characteristic peaks at 2theta values of 17.3°±0.2° and 18.2°±0.2° using CuKα radiation. Preferably, the X-ray powder diffraction pattern of Form CSI comprises two characteristic peaks at 2theta values of 17.3°±0.2° and 18.2°±0.2° using CuKα radiation.

In another aspect provided herein, the X-ray powder diffraction pattern of Form CSI comprises three or four or five or six or seven or eight characteristic peaks at 2theta values of 3.2°±0.2°, 5.6°±0.2°, 8.6°±0.2°, 11.8°±0.2°, 14.2°±0.2°, 15.0°±0.2°, 17.3°±0.2° and 18.2°±0.2° using CuKα radiation using CuKα radiation.

Without any limitation being implied, the X-ray powder diffraction pattern of Form CSI is substantially as depicted in FIG. 1.

Without any limitation being implied, Form CSI is a hydrate.

Without any limitation being implied, the Thermo Gravimetric Analysis (TGA) curve of Form CSI is substantially as depicted in FIG. 3, which shows 7.6% weight loss when heated to 200° C.

Without any limitation being implied, the Differential Scanning calorimetry (DSC) curve of Form CSI is substantially as depicted in FIG. 4, which shows an endothermic peak at around 108° C. (onset temperature), which is a dehydration endothermic peak, an exothermic peak at around 137° C. (onset temperature), and another endothermic peak at around 263° C. (onset temperature).

According to the objective of the present disclosure, a process for preparing Form CSI is also provided. The process comprises: adding Compound I solid in water, the solvent mixture of water and an alcohol, water and a ketone, water and a nitrile, or water and an ether, stirring, separating and drying to obtain Form CSI.

Furthermore, said alcohol is preferably a C1-C8 alcohol, said ketone is preferably a C3-C6 ketone, said nitrile is preferably a C2-C4 nitrile, said ether is preferably a C2-C7 ether.

Furthermore, said alcohol is preferably ethanol, said ketone is preferably acetone, said nitrile is preferably acetonitrile, said ether is preferably 1,4-dioxane; said volume ratio of water in the mixture solvent is preferably 15%-100%; said stirring time is preferably at least one day; said stirring temperature is preferably 4° C.-50° C., further preferably room temperature.

Form CSI of the present disclosure has the following advantages:

(1) Compared with prior arts, Form CSI of the present disclosure has better stability. Without any limitation being implied, in a specific example provided by the present disclosure, the mixture of Form CSI and prior art Form A was suspended in a solvent, stirred at 5° C. and Form CSI was obtained, which indicates that Form CSI has better stability.

(2) Form CSI drug substance of the present disclosure has good stability. Crystalline state of Form CSI drug substance doesn't change for at least 6 months when stored under 25° C./60% relative humidity (RH) with open and sealed condition. The chemical purity is above 98.0% and remains substantially unchanged during storage. These results show that Form CSI drug substance has good stability under long-term storage condition, which is beneficial to the drug storage.

Meanwhile, crystalline state of Form CSI drug substance doesn't change for at least 6 months when stored under 40° C./75% RH with open and sealed condition. The chemical purity remains substantially unchanged during storage. The results show that Form CSI drug substance has good stability under accelerated condition. Drug substance will go through high temperature and high humidity conditions caused by different season, regional climate and environment during storage, transportation, and manufacturing processes. Therefore, good stability under accelerated and stress conditions is of great importance to the drug development. Form CSI drug substance has good stability under stress condition, which is beneficial to avoid the influence on drug quality when not stored under condition recommended in label.

Meanwhile, Form CSI has good mechanical stability. Crystalline state of Form CSI drug substance doesn't change after tableting, which shows that Form CSI has good physical stability. Form CSI drug substance has good physical stability under different pressures, which is beneficial to keep crystalline form stable during tableting process.

Crystal transformation can lead to changes in the absorption of the drug, affect bioavailability, and even cause toxicity and side effects. Good chemical stability of drug substance ensure that no impurities are generated during production and storage. Form CSI has good physical stability, ensuring consistent and controllable quality of the drug substance and drug product, minimizing quality change, bioavailability change and toxicity due to crystal transformation or impurity generation.

(3) Compared with prior arts, Form CSI of the present disclosure shows superior adhesiveness. Adhesiveness evaluation results indicate that adhesion quantity of Form CSI is lower than that of Form A. Due to the superior adhesiveness of Form CSI, adhesion to roller and tooling during dry-granulation and compression process can be reduced, which is beneficial to improve product appearance and weight variation. In addition, superior adhesiveness of Form CSI can reduce the agglomeration of drug substance, which is beneficial to the dispersion of drug substance and reduce the adhesion between drug substance and instruments, improves the blend uniformity, and content uniformity of drug product.

(4) Compared with prior arts, Form CSI of the present disclosure has better compressibility. Failure in hardness/friability test and tablet crack issue can be avoided due to better compressibility of Form CSI, making the preparation process more reliable, improving product appearance and product quality. Better compressibility can increase the compression rate, thus further increases the efficiency of process and reduces the cost of compressibility improving excipients.

According to the objective of the present disclosure, crystalline form CSII of Compound I is provided (hereinafter referred to as “Form CSII”).

Without any limitation being implied, Form CSII has different states under different humidity conditions.

In one aspect provided herein, when the relative humidity is approximately below 30%, the X-ray powder diffraction pattern of Form CSII comprises characteristic peaks at 2theta values of 4.0°±0.2°, 11.4°±0.2° and 13.5°±0.2° using CuKα radiation.

Furthermore, when the relative humidity is approximately below 30%, the X-ray powder diffraction pattern of Form CSII comprises one or two or three characteristic peaks at 2theta values of 7.4°±0.2°, 8.7°±0.2° and 12.0°±0.2° using CuKα radiation. Preferably, the X-ray powder diffraction pattern of Form CSII comprises three characteristic peaks at 2theta values of 7.4°±0.2°, 8.7°±0.2° and 12.0°±0.2° using CuKα radiation.

Furthermore, when the relative humidity is approximately below 30%, the X-ray powder diffraction pattern of Form CSII comprises one or two or three characteristic peaks at 2theta values of 17.5°±0.2°, 20.9°±0.2° and 24.0°±0.2° using CuKα radiation. Preferably, the X-ray powder diffraction pattern of Form CSII comprises three characteristic peaks at 2theta values of 17.5°±0.2°, 20.9°±0.2° and 24.0°±0.2° CuKα radiation.

In another aspect provided herein, when the relative humidity is approximately below 30%, the X-ray powder diffraction pattern of Form CSII comprises three or four or five or six or seven or eight or nine or ten or eleven or twelve characteristic peaks at 2theta values of 4.0°±0.2°, 11.4°±0.2°, 13.5°±0.2°, 8.1°±0.2°, 7.4°±0.2°, 8.7°±0.2°, 12.0°±0.2°, 14.9°±0.2°, 16.2°±0.2°, 17.5°±0.2°, 20.9°±0.2° and 24.0°±0.2° using CuKα radiation.

Without any limitation being implied, when the relative humidity is approximately below 30%, the X-ray powder diffraction pattern of Form CSII is substantially as depicted in FIG. 5.

Without any limitation being implied, when the relative humidity is approximately below 30%, the TGA curve of Form CSII is substantially as depicted in FIG. 6, which shows 0.3% weight loss when heated to 200° C.

In another aspect provided herein, when the relative humidity is approximately above 30%, the X-ray powder diffraction pattern of Form CSII comprises characteristic peaks at 2theta values of 3.8°±0.2°, 7.7°±0.2° and 12.1°±0.2° using CuKα radiation.

Furthermore, when the relative humidity is approximately above 30%, the X-ray powder diffraction pattern of Form CSII comprises one or two or three characteristic peaks at 2theta values of 9.4°±0.2°, 15.2°±0.2° and 18.9°±0.2° using CuKα radiation. Preferably, the X-ray powder diffraction pattern of Form CSII comprises three characteristic peaks at 2theta values of 9.4°±0.2°, 15.2°±0.2° and 18.9°±0.2° using CuKα radiation.

Furthermore, when the relative humidity is approximately above 30%, the X-ray powder diffraction pattern of Form CSII comprises one or two or three characteristic peaks at 2theta values of 11.4°±0.2°, 15.4°±0.2° and 22.9°±0.2° using CuKα radiation. Preferably, the X-ray powder diffraction pattern of Form CSII comprises three characteristic peaks at 2theta values of 11.4°±0.2°, 15.4°±0.2° and 22.9°±0.2° using CuKα radiation.

In another aspect provided herein, when the relative humidity is approximately above 30%, the X-ray powder diffraction pattern of Form CSII comprises three or four or five or six or seven or eight or nine characteristic peaks at 2theta values of 3.8°±0.2°, 7.7°±0.2°, 12.1°±0.2°, 9.4°±0.2°, 15.2°±0.2°, 18.9°±0.2°, 11.4°±0.2°, 15.4°±0.2° and 22.9°±0.2° using CuKα radiation.

Without any limitation being implied, when the relative humidity is approximately above 30%, the X-ray powder diffraction pattern of Form CSII is substantially as depicted in FIG. 8.

According to the objective of the present disclosure, a process for preparing Form CSII is also provided. The process comprises: dissolving Compound I solid into a solvent mixture of water, an ether, and an alcohol or a solvent mixture of water, an ether, and a ketone, volatilizing and drying to obtain Form CSII.

Furthermore, said alcohol is preferably a C1-C8 alcohol, said ether is preferably a C2-C7 ether, said ketone is preferably a C3-C6 ketone, said volume ratio of water in the mixture is preferably 2%-10%, said volume ratio of alcohol or ketone is preferably 2%-40%.

Furthermore, said alcohol is preferably ethanol, said ether is preferably tetrahydrofuran, said ketone is preferably acetone.

Form CSII of the present disclosure has the following advantages:

(1) Form CSII drug substance of the present disclosure has good stability.

Crystalline state of Form CSII drug substance doesn't change for at least 6 months when stored under 25° C./60% RH with open and sealed condition. The chemical purity remains substantially unchanged during storage. These results show that Form CSII drug substance has good stability under long-term storage condition, which is beneficial to the drug storage.

Meanwhile, crystalline state of Form CSII drug substance doesn't change for at least 6 months when stored under 40° C./75% RH with open and sealed condition. The chemical purity remains substantially unchanged during storage. These results show that Form CSII drug substance has good stability under accelerated condition. Drug substance will go through high temperature and high humidity conditions caused by different season, regional climate and environment during storage, transportation, and manufacturing processes. Therefore, good stability under accelerated and stress conditions is of great importance to the drug development. Form CSII drug substance has good stability under stress condition, which is beneficial to avoid the influence on drug quality when not stored in condition recommended in label.

Meanwhile, Form CSII has good mechanical stability. Crystalline state of Form CSII drug substance doesn't change after grinding, which shows that Form CSII has good physical stability. Grinding and pulverization are often required in the drug manufacturing process. Good physical stability of the drug substance can reduce the risk of crystallinity decrease and crystal transformation during the drug production process. Form CSII drug substance has good physical stability under different pressures, which is beneficial to keep crystalline form stable during tableting process.

Crystal transformation can lead to changes in the absorption of the drug, affect bioavailability, and even cause toxicity and side effects. Good chemical stability of drug substance ensure that no impurities are generated during production and storage. Form CSII has good physical stability, ensuring consistent and controllable quality of the drug substance and drug product, minimizing quality change, bioavailability change and toxicity due to crystal transformation or impurity generation.

(2) Compared with prior arts, Form CSII of the present disclosure shows superior adhesiveness. Adhesiveness evaluation results indicate that adhesion quantity of Form A is 3.2 times that of Form CSII. The adhesion quantity of Form CSII is much lower than that of Form A. Due to the superior adhesiveness of Form CSII, adhesion to roller and tooling during dry-granulation and compression process can be reduced, which is beneficial to improve product appearance and weight variation. In addition, superior adhesiveness of Form CSII can reduce the agglomeration of drug substance and the adhesion between drug substance and instruments, which is beneficial to the dispersion of drug substance and the mixing with other excipients, and to improve the uniformity when the materials are mixed and the content uniformity of the final drug product.

(3) Compared with prior arts, Form CSII of the present disclosure has better compressibility. Failure in hardness/friability test and tablet crack issue can be avoided due to better compressibility of Form CSII, making the preparation process more reliable, improving product appearance and product quality. Better compressibility can increase the compression rate, thus further increases the efficiency of process and reduces the cost of compressibility improving excipients.

According to the objective of the present disclosure, a pharmaceutical composition is provided, said pharmaceutical composition comprises a therapeutically effective amount of Form CSI or Form CSII or combinations thereof and pharmaceutically acceptable excipients.

Furthermore, Form CSI or Form CSII or combinations thereof can be used for preparing TYK2 inhibitor drugs.

Furthermore, Form CSI or Form CSII or combinations thereof can be used for preparing drugs treating psoriasis, systemic lupus erythematosus, and Crohn's disease.

In the present disclosure, said “stirring” is accomplished by using a conventional method in the field such as magnetic stirring or mechanical stirring and the stirring speed is 50 to 1800 r/min, preferably the magnetic stirring speed is 300 to 900 r/min and mechanical stirring speed is 100 to 300 r/min.

Said “drying” is accomplished at room temperature or a higher temperature. The drying temperature is from room temperature to about 100° C., or to 60° C., or to 50° C., or to 40° C. The drying time can be 0.5 to 48 hours, or overnight. Drying is accomplished in a fume hood, forced air convection oven or vacuum oven.

Said “separation” is accomplished by using a conventional method in the field such as centrifugation or filtration. The operation of “centrifugation” is as follows: the sample to be separated is placed into the centrifuge tube, and then centrifuged at a rate of 10000 r/min until the solid all sink to the bottom of the tube.

Said “evaporating” is accomplished by using a conventional method in the field such as slow evaporation or rapid evaporation. Slow evaporation is accomplished in a container covered by a sealing film with pinholes. Rapid evaporation is accomplished in an open container.

Said “rotary evaporation” is accomplished by using a conventional method in the field. For example, the operation of rotary evaporation is to rotate the flask containing solution at a constant speed at a certain temperature and a certain reduced pressure to evaporate the solvent.

Said “characteristic peak” refers to a representative diffraction peak used to distinguish crystals, which usually can have a deviation of ±0.2° using CuKα radiation.

In the present disclosure, “crystal” or “crystalline form” refers to the crystal or the crystalline form being identified by the X-ray diffraction pattern shown herein. Those skilled in the art are able to understand that the experimental errors depend on the instrument conditions, the sample preparation and the purity of samples. The relative intensity of the diffraction peaks in the X-ray diffraction pattern may also vary with the experimental conditions; therefore, the order of the diffraction peak intensities cannot be regarded as the sole or decisive factor. In fact, the relative intensity of the diffraction peaks in the X-ray powder diffraction pattern is related to the preferred orientation of the crystals, and the diffraction peak intensities shown herein are illustrative and identical diffraction peak intensities are not required. Thus, it will be understood by those skilled in the art that a crystalline form of the present disclosure is not necessarily to have exactly the same X-ray diffraction pattern of the example shown herein. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms.

In some embodiments, Form CSI or Form CSII of the present disclosure is pure and substantially free of any other crystalline forms. In the present disclosure, the term “substantially free” when used to describe a novel crystalline form, it means that the content of other crystalline forms in the novel crystalline form is less than 20% (w/w), specifically less than 10% (w/w), more specifically less than 5% (w/w) and furthermore specifically less than 1% (w/w).

In the present disclosure, the term “about” when referring to a measurable value such as weight, time, temperature, and the like, is meant to encompass variations of ±10%, ±5%, ±1%, ±0.5%, or even ±0.1% of the specified amount.

### BRIEF DESCRIPTION OF THE DRAWINGS

FIG. 1 shows an XRPD pattern of Form CSI in Example 1.

FIG. 2 shows an XRPD pattern of Form CSI in Example 2.

FIG. 3 shows a TGA curve of Form CSI in Example 3.

FIG. 4 shows a DSC curve of Form CSI in Example 3.

FIG. 5 shows an XRPD pattern of Form CSII in Example 4.

FIG. 6 shows a TGA curve of Form CSII in Example 4.

FIG. 7 shows a DSC curve of Form CSII in Example 4.

FIG. 8 shows an XRPD pattern of Form CSII in Example 5.

FIG. 9 shows an XRPD pattern overlay of Form CSI and Form A before and after stirring at a solvent mixture of water and acetonitrile (v/v, 1:9) (from top to bottom: initial Form A, initial Form CSI, stirring for 5 minutes, stirring for 6 days).

FIG. 10 shows an XRPD pattern overlay of Form CSI before and after storage under different conditions for 6 months (from top to bottom: initial, 25° C./60% RH (sealed), 25° C./60% RH (open), 40° C./75% RH (sealed), 40° C./75% RH (open)).

FIG. 11 shows an XRPD pattern overlay of Form CSI before and after tableting under different pressures (from top to bottom: initial, tableting under 3 kN, tableting under 7 kN, tableting under 14 kN).

FIG. 12 shows an XRPD pattern overlay of Form CSII before and after storage under different conditions for 6 months (from top to bottom: initial, 25° C./60% RH (sealed), 25° C./60% RH (open), 40° C./75% RH (sealed), 40° C./75% RH (open)).

FIG. 13 shows an XRPD pattern overlay of Form CSII before and after tableting under different pressures (from top to bottom: initial, tableting under 5 kN, tableting under 10 kN, tableting under 20 kN).

FIG. 14 shows an XRPD pattern overlay of Form CSII before and after grinding (top: after grinding, bottom: before grinding).

### DETAILED DESCRIPTION

The present disclosure is further illustrated by the following examples which describe the preparation and use of the crystalline forms of the present disclosure in detail. It is obvious to those skilled in the art that many changes in the materials and methods can be accomplished without departing from the scope of the present disclosure.

The abbreviations used in the present disclosure are explained as follows:

XRPD: X-ray Powder Diffraction

DSC: Differential Scanning calorimetry

TGA: Thermo Gravimetric Analysis

¹H NMR: Proton Nuclear Magnetic Resonance

HPLC: High Performance Liquid Chromatography

Instruments and Methods Used for Data Collection:

X-ray powder diffraction patterns in the present disclosure examples 1-5 were acquired by a Bruker D2 PHASER X-ray powder diffractometer. The parameters of the X-ray powder diffraction method of the present disclosure are as follows:

X-ray source: Cu, Kα

### Kα1 (Å): 1.54060; Kα2 (Å): 1.54439

Kα2/Kα1 intensity ratio: 0.50

### Voltage: 30 (kV)

### Current: 10 (mA)

Scan range: from 3.0 degree to 40.0 degree

X-ray powder diffraction patterns in the present disclosure examples 6-8 and 11-12 were acquired by Bruker D8 DISCOVER X-ray powder diffractometer. The parameters of the X-ray powder diffraction method of the present disclosure are as follows:

X-ray source: Cu, Kα

### Kα1 (Å): 1.54060; Kα2 (Å): 1.54439

Kα2/Kα1 intensity ratio: 0.50

### Voltage: 40 (kV)

### Current: 40 (mA)

Scan range: from 4.0 degree to 40.0 degree

Differential scanning calorimetry (DSC) data in the present disclosure were acquired by a TA Q2000. The parameters of the DSC method of the present disclosure are as follows:

Heating rate: 10° C./min
Purge gas: nitrogen

Thermo gravimetric analysis (TGA) data in the present disclosure were acquired by a TA Q500. The parameters of the TGA method of the present disclosure are as follows:

Heating rate: 10° C./min
Purge gas: nitrogen

Proton nuclear magnetic resonance spectrum data (¹H NMR) were collected from a Bruker Avance II DMX 400M Hz NMR spectrometer. 1-5 mg of sample was weighed and dissolved in 0.5 mL of deuterated chloroform to obtain a solution with a concentration of 2-10 mg/mL.

The parameters for related substance detection in the present disclosure are shown in Table 1.

TABLE 1

| HPLC | Waters ACQUITY H-class | |
|---|---|---|
| | UPLC with PDA detector | |
| Column | Waters ACQUITY UPLC BEH | |
| | C18, 2.1 × 50 mm, 1.7 μm | |
| Mobile Phase | A: 0.1% Trifluoroacetic acid | |
| | (TFA) in H₂O (v/v) | |
| | B: 0.1% TFA in Acetonitrile | |
| | (v/v) | |
| Gradient | Time (min) | % B |
| | 0.0 | 10 |
| | 0.5 | 10 |
| | 7.0 | 80 |
| | 8.0 | 80 |
| | 8.1 | 10 |
| | 10.0 | 10 |
| Running time | 10.0 | min |
| Stop time | 0 | min |
| Speed | 0.5 | mL/min |
| Injection Volume | 1 | μL |
| Detection wavelength | UV, 240 | nm |
| Column Temperature | 40° | C. |

| | |
|---|---|
| Sample Temperature | Room Temperature |
| Diluent | Acetonitrile: H₂O = 50:50 (v/v) |

Unless otherwise specified, the following examples were conducted at room temperature. Said “room temperature” is not a specific temperature, but a temperature range of 10-30° C.

According to the present disclosure, Compound I used as a raw material includes but not limited to solid (crystalline or amorphous), oil, liquid and solution. Preferably, Compound I and/or its salts used as the raw material is a solid.

Compound I used in the following examples can be prepared by known methods in the prior arts, for example, the method disclosed in WO2018183656A1.

### EXAMPLES

### Example 1 Preparation of Form CSI

86.1 mg of Compound I solid was weighed into a glass vial, and then 0.8 mL of water was added. The system was stirred at room temperature for 8 days. A solid was obtained by separating and drying.

The solid was confirmed to be Form CSI. The XRPD pattern of Form CSI is shown in FIG. 1, and the XRPD data are listed in Table 2.

TABLE 2

| Diffraction | | |
|---|---|---|
| angle 2θ | d spacing | Intensity % |
| 3.20 | 27.64 | 100.00 |
| 5.62 | 15.73 | 57.58 |
| 6.44 | 13.71 | 8.70 |
| 8.59 | 10.30 | 98.62 |
| 9.75 | 9.07 | 3.76 |
| 11.75 | 7.53 | 21.95 |
| 14.23 | 6.22 | 18.21 |
| 14.98 | 5.91 | 16.16 |
| 16.28 | 5.44 | 2.58 |
| 17.25 | 5.14 | 7.63 |
| 18.17 | 4.88 | 10.16 |
| 19.84 | 4.48 | 4.54 |
| 21.47 | 4.14 | 4.27 |
| 23.60 | 3.77 | 12.17 |
| 26.08 | 3.42 | 5.11 |

### Example 2 Preparation of Form CSI

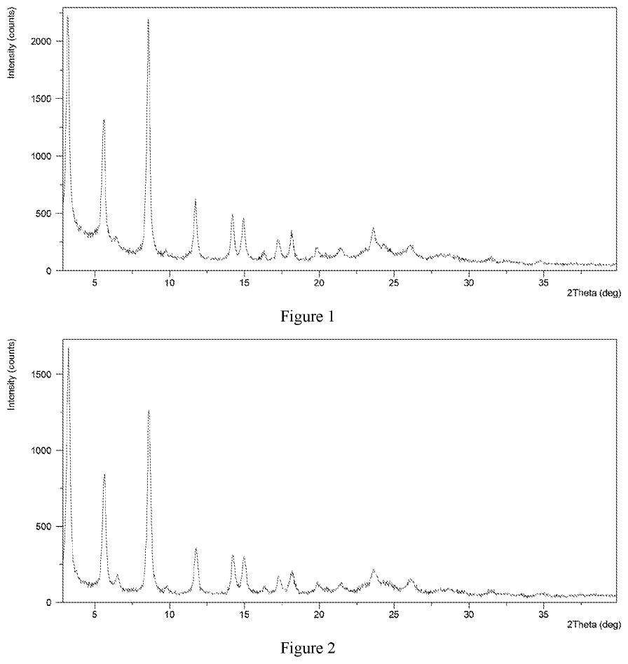

230.8 mg of Compound I solid was weighed and dissolved into 13 mL of chloroform to obtain a clear solution. The solution was filtered, and the filtrate was rotary evaporated at 40° C. to obtain a dry solid. A certain amount of the dried solid was weighed into a vial, and then a certain volume of solvent was added according to Table 3. The system was stirred at room temperature for a certain period, filtered and dried to obtain a solid. Samples 1˜4 were confirmed to be Form CSI. The XRPD pattern of Sample 2 is shown in FIG. 2, and the XRPD data are listed in Table 4.

TABLE 3

| | Weight of | | Vol- | Stirring | Crystal- |
|---|---|---|---|---|---|
| Num- | the dried | | ume | time | line |
| ber | solid (mg) | Solvent (v:v) | (mL) | (day) | form |
| 1 | 12.0 | water | 0.30 | 1 | CSI |
| 2 | 20.7 | water/ethanol | 0.35 | 4 | CSI |
| | | (6:4) | | | |
| 3 | 17.8 | water/acetone | 0.35 | 4 | CSI |
| | | (4:6) | | | |
| 4 | 17.9 | water/1,4- | 0.30 | 4 | CSI |
| | | dioxane (1:1) | | | |

TABLE 4

| Diffraction | | |
|---|---|---|
| angle 2θ | d spacing | Intensity % |
| 3.26 | 27.11 | 100.00 |
| 5.69 | 15.54 | 45.27 |
| 6.53 | 13.54 | 7.53 |
| 8.59 | 10.30 | 74.67 |
| 9.84 | 8.99 | 2.91 |
| 11.75 | 7.53 | 17.73 |
| 14.21 | 6.23 | 15.79 |
| 15.00 | 5.91 | 15.17 |
| 16.38 | 5.41 | 1.97 |
| 17.29 | 5.13 | 7.05 |
| 18.24 | 4.86 | 7.58 |
| 19.91 | 4.46 | 3.77 |
| 21.46 | 4.14 | 3.81 |
| 23.64 | 3.76 | 9.46 |
| 24.69 | 3.61 | 3.95 |
| 26.19 | 3.40 | 4.96 |

### Example 3 Preparation of Form CSI

306.6 mg of Compound I solid was weighed into a glass vial, and 15 mL of water was added. The system was stirred at room temperature for 10 days and filtered to obtain a solid. The obtained solid was dried under vacuum at room temperature for 9 hours. The solid was confirmed to be Form CSI.

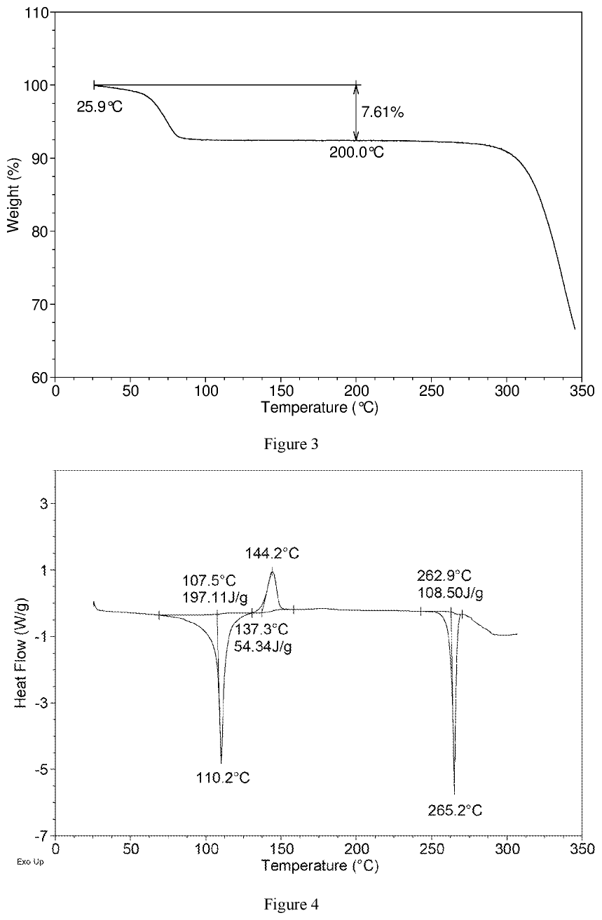

The TGA curve of Form CSI is substantially as depicted in FIG. 3, which shows about 7.6% weight loss when heated to 200° C.

The DSC curve of Form CSI is substantially as depicted in FIG. 4. The DSC curve shows the first endothermic peak at around 108° C. (onset temperature) corresponding to dehydration, an exothermic peak at around 137° C. (onset temperature), and the second endothermic peak at around 263° C. (onset temperature).

The ¹H NMR data of Form CSI are: ¹H NMR (400 MHz, CDCl₃) δ 10.97 (s, 1H), 9.44 (s, 1H), 8.20 (s, 1H), 8.10 (s, 1H), 8.05 (s, 1H), 7.79 (dd, J=7.9, 1.6 Hz, 1H), 7.50 (dd, J=8.0, 1.5 Hz, 1H), 7.25 (t, J=7.9 Hz, 1H), 4.00 (s, 3H), 3.80 (s, 3H), 1.84-1.75 (m, 1H), 1.14-1.05 (m, 2H), 0.93-0.84 (m, 2H).

### Example 4 Preparation of Form CSII

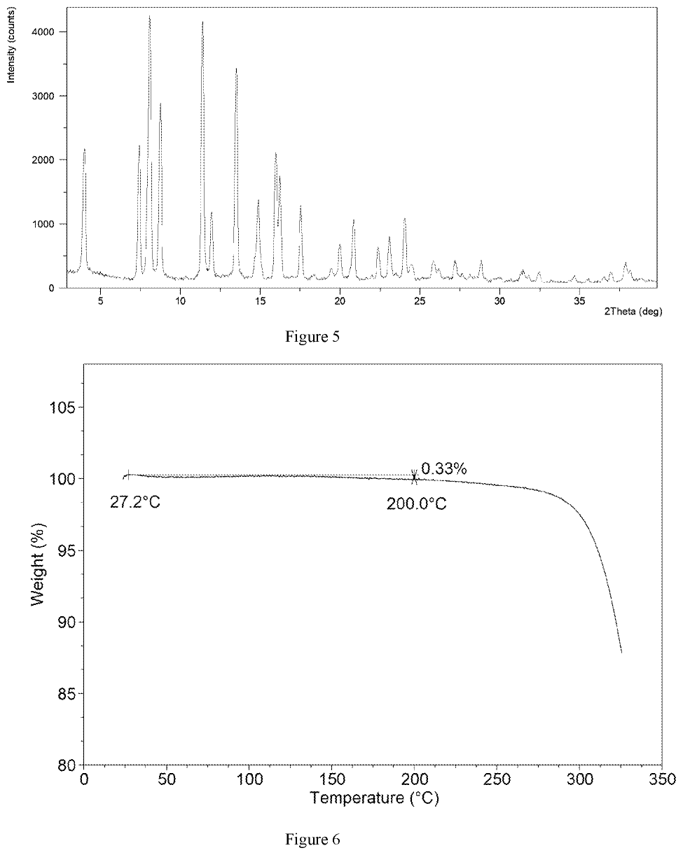

A certain amount of Compound I solid was weighed and dissolved into a certain volume of solvent according to Table 5 to form a solution. The solution was evaporated at room temperature for one day to obtain a solid. The solid was dried under vacuum at 35° C. for two days, and then dried under vacuum at 80° C. for 3.5 hours. Samples 1-5 were confirmed to be Form CSII. The XRPD pattern of Sample 3 is shown in FIG. 5, and the XRPD data are listed in Table 6.

The TGA curve of Form CSII is substantially as depicted in FIG. 6, which shows about 0.3% weight loss when heated to 200° C.

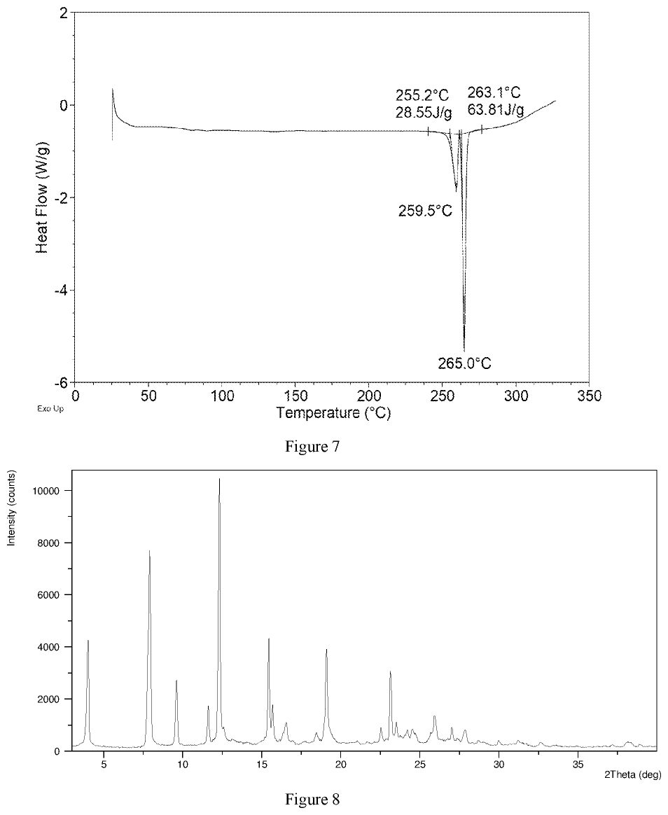

The DSC curve of Form CSII is substantially as depicted in FIG. 7, which shows the first endothermic peak at 259.5° C. (onset temperature), which is the melting endotherm peak.

TABLE 5

| | Weight of | | | Crystal- |
|---|---|---|---|---|
| Num- | Compound | | Volume | line |
| ber | I (mg) | Solvent (v:v:v) | (mL) | form |
| 1 | 9.5 | water/ | 1 | CSII |
| | | tetrahydrofuran/ | | |
| | | acetone (1:20:1) | | |
| 2 | 9.8 | water/ | 1 | CSII |
| | | tetrahydrofuran/ | | |
| | | acetone (1:15:1) | | |
| 3 | 9.9 | water/ | 1 | CSII |
| | | tetrahydrofuran/ | | |
| | | acetone (1:15:5) | | |
| 4 | 9.3 | water/ | 1 | CSII |
| | | tetrahydrofuran/ | | |
| | | ethanol (1:20:1) | | |
| 5 | 10.4 | water/ | 1 | CSII |
| | | tetrahydrofuran/ | | |
| | | ethanol (1:15:1) | | |

TABLE 6

| Diffraction | | |
|---|---|---|
| angle 2θ | d spacing | Intensity % |
| 3.98 | 22.18 | 48.40 |
| 7.43 | 11.89 | 50.29 |
| 8.08 | 10.94 | 100.00 |
| 8.74 | 10.11 | 66.90 |
| 11.40 | 7.77 | 98.70 |
| 11.96 | 7.40 | 25.38 |
| 13.50 | 6.56 | 80.28 |
| 14.88 | 5.96 | 30.47 |
| 15.97 | 5.55 | 47.52 |
| 16.23 | 5.46 | 39.04 |
| 17.54 | 5.06 | 28.54 |
| 19.44 | 4.57 | 4.25 |
| 20.00 | 4.44 | 13.46 |
| 20.85 | 4.26 | 23.07 |
| 22.39 | 3.97 | 12.84 |
| 23.10 | 3.85 | 16.93 |
| 24.05 | 3.70 | 23.81 |
| 24.48 | 3.64 | 6.28 |
| 25.85 | 3.45 | 7.80 |
| 26.19 | 3.40 | 4.55 |
| 27.20 | 3.28 | 6.97 |
| 28.84 | 3.10 | 7.95 |
| 31.47 | 2.84 | 3.88 |
| 32.46 | 2.76 | 3.70 |
| 36.95 | 2.43 | 3.90 |
| 37.89 | 2.37 | 7.12 |
| 38.16 | 2.36 | 4.29 |

### Example 5 Preparation of Form CSII

Form CSII was exposed to a high relative humidity condition for a certain time, the XRPD pattern of Form CSII is substantially as depicted in FIG. 8, and the XRPD data are listed in Table 7.

The ¹H NMR data of Form CSII are: ¹H NMR (400 MHz, CDCl₃) δ 10.98 (s, 1H), 9.98 (s, 1H), 8.24 (s, 1H), 8.11 (s, 1H), 8.04 (s, 1H), 7.80 (dd, J=7.8, 1.2 Hz, 1H), 7.52 (dd, J=7.9, 1.0 Hz, 1H), 7.26 (t, J=7.9 Hz, 1H), 4.00 (s, 3H), 3.81 (s, 3H), 1.92-1.84 (m, 1H), 1.13-1.07 (m, 2H), 0.92-0.85 (m, 2H).

TABLE 7

| Diffraction | | |
|---|---|---|
| angle 2θ | d spacing | Intensity % |
| 3.77 | 23.42 | 40.82 |
| 7.68 | 11.51 | 73.93 |
| 9.38 | 9.43 | 24.87 |
| 11.38 | 7.78 | 14.64 |
| 12.08 | 7.33 | 100.00 |
| 15.20 | 5.83 | 40.02 |
| 15.45 | 5.74 | 14.78 |
| 16.26 | 5.45 | 7.25 |
| 18.22 | 4.87 | 3.81 |
| 18.86 | 4.71 | 35.48 |
| 22.31 | 3.99 | 6.02 |
| 22.90 | 3.88 | 26.07 |
| 23.27 | 3.82 | 8.13 |
| 24.29 | 3.66 | 5.59 |
| 25.70 | 3.47 | 10.75 |
| 26.78 | 3.33 | 6.27 |
| 27.61 | 3.23 | 5.59 |
| 31.06 | 2.88 | 1.34 |
| 32.43 | 2.76 | 1.09 |
| 37.96 | 2.37 | 1.55 |

### Example 6 Thermodynamic Stability of Form CSI

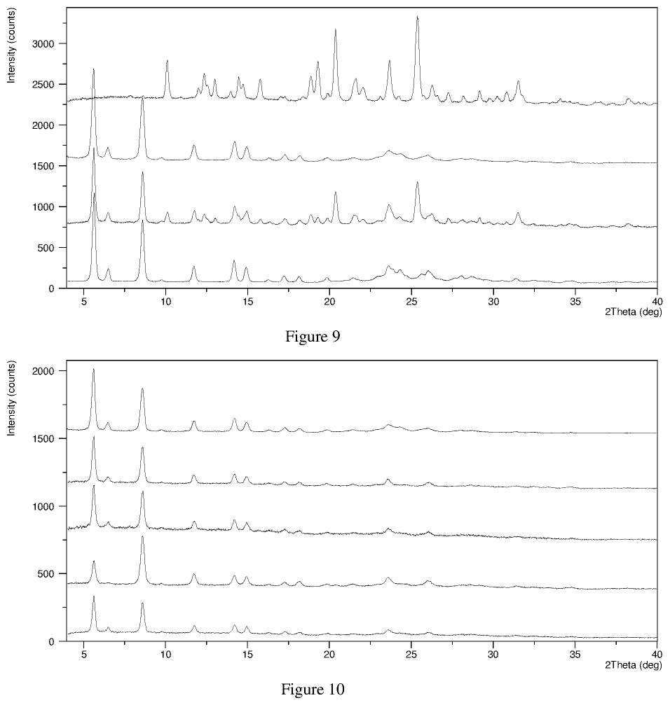

A certain amount of Form A was weighed into glass vail and 0.3 mL of solvent mixtures of water and acetonitrile, water and methanol, or water and acetone according to table 8 were added to form suspensions. Approximately 6 mg of Form CSI of the present disclosure was added in the suspension. After mixing at 5° C. for 5 minutes, a portion of the solid was taken out and tested by XRPD. After the suspension was stirred at 5° C. for 6 days, a portion of the solid was taken out and tested by XRPD again. The results are shown in Table 8. The XRPD overlay of Sample 1 before and after stirring in water/acetonitrile is shown in FIG. 9.

TABLE 8

| Sam- | Weight of | | Stirring time | |
|---|---|---|---|---|
| ple | Form A (mg) | Solvent (v/v) | 5 minutes | 6 days |
| 1 | 5.0 | water/acetonitrile | Form A + | Form |
| 2 | 4.6 | water/acetonitrile | Form | CSI |
| 3 | 6.4 | water/methanol (5:5) | CSI | |
| 4 | 5.9 | water/methanol (7:3) | | |
| 5 | 6.5 | water/acetone (2:8) | | |
| 6 | 6.4 | water/acetone (3:7) | | |
| 7 | 5.4 | water/acetone (7:3) | | |

The results show that Form CSI has better stability than Form A in the solvent systems of water/acetonitrile, water/methanol or water/acetone with a certain volume ratio under 5° C. condition.

### Example 7 Stability of Form CSI

Approximately 5 mg of Form CSI in the present disclosure was stored under different conditions of 25° C./60% RH and 40° C./75% RH. Crystalline form and chemical purity were checked by XRPD and HPLC, respectively. The results are shown in Table 9, and the XRPD overlay is shown in FIG. 10.

TABLE 9

| | | | Crystal- | |
|---|---|---|---|---|
| Initial | | | line | |
| form | Conditions | Time | form | Purity |
| Form | Initial | — | Form | 98.13% |
| CSI | 25^{o} C./60% RH (open) | 6 months | CSI | 98.76% |
| | 25^{o} C./60% RH (sealed) | 6 months | | 98.79% |
| | 40^{o} C./75% RH (open) | 6 months | | 98.74% |
| | 40^{o} C./75% RH (sealed) | 6 months | | 98.73% |

The results show that Form CSI is stable for at least 6 months at 25° C./60% RH and 40° C./75% RH. Form CSI has good stability under long-term and accelerated conditions.

### Example 8 Physical Stability of Form CSI Under Pressure

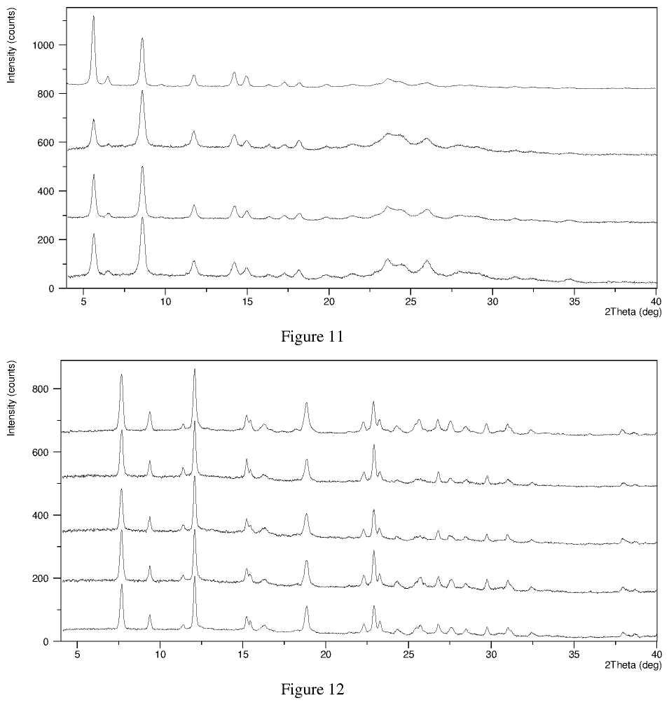

A certain amount of Form CSI was compressed into tablets under 3 kN, 7 kN and 14 kN with suitable tableting die. Crystalline forms before and after tableting were checked by XRPD. The test results show that crystalline state of Form CSI does not change under different pressures. The XRPD overlay is shown in FIG. 11.

### Example 9 Compressibility of Form CSI

ENERPAC manual tablet press was used for compression. 80 mg of Form CSI and Form A were weighed and added into the dies of a φ6 mm round tooling, compressed at 10 KN manually, then stored at room temperature for 24 h until complete elastic recovery. Hardness (H) was tested with an intelligent tablet hardness tester. Diameter (D) and thickness (L) were tested with caliper. Tensile strength of the powder was calculated with the following formula: T=2H/πDL*9.8. Under a certain force, the greater the tensile strength, the better the compressibility. The results are presented in Table 10. The results indicate that Form A is splintering during the tablet pressing process, Form CSI has better compressibility compared with Form A.

TABLE 10

| | Thickness | Diameter | Hardness | Tensile |
|---|---|---|---|---|
| Form | (mm) | (mm) | (kgf) | strength (MPa) |
| Form A | N/A | N/A | N/A | N/A |
| Form CSI | 2.39 | 6.06 | 7.87 | 3.39 |

### Example 10 Adhesiveness of Form CSI

Approximately 30 mg of Form CSI and Form A were weighed and added into the dies of φ8 mm round tooling, compressed at 10 kN and held for 30 s. The material sticking to the punch was recorded. The compression was repeated twice and the cumulative amount, maximum amount and average amount of material sticking to the punch during the compression process were recorded. The results are shown in Table 11. The results indicate that the adhesiveness of Form CSI is superior to that of Form A.

TABLE 11

| | Maximum | Average |
|---|---|---|
| Form | amount (mg) | amount (mg) |
| Form A | 0.260 | 0.190 |
| Form CSI | 0.130 | 0.105 |

### Example 11 Stability of Form CSII

Approximately 5 mg of Form CSII in the present disclosure was stored under different conditions of 25° C./60% RH and 40° C./75% RH. Crystalline form and chemical purity were checked by XRPD and HPLC, respectively. The results are shown in Table 12, and the XRPD overlay is shown in FIG. 12.

TABLE 12

| Initial | | | Crystalline |
|---|---|---|---|
| form | Conditions | Time | form |
| Form | Initial | — | Form CSII |
| CSII | 25^{o} C./60% RH (open) | 6 months | |
| | 25^{o} C./60% RH (closed) | 6 months | |
| | 40^{o} C./75% RH (open) | 6 months | |
| | 40^{o} C./75% RH (closed) | 6 months | |

The results show that Form CSII is stable for at least 6 months at 25° C./60% RH and 40° C./75% RH. Form CSII has good stability under long-term and accelerated conditions.

### Example 12 Physical Stability of Form CSII Under Pressure

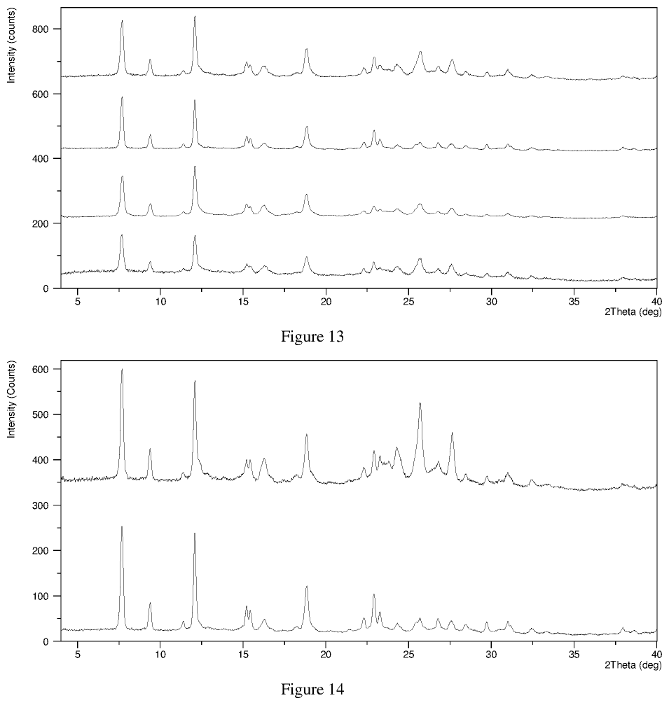

A certain amount of Form CSII was compressed into tablets under 5 kN, 10 kN and 20 kN with suitable tableting die. Crystalline forms before and after tableting were checked by XRPD. The test results show that crystalline state of Form CSII does not change under different pressures. The XRPD overlay is shown in FIG. 13.

Form CSII was grounded manually for 5 minutes in a mortar. Crystalline form before and after grinding were checked by XRPD. The test results show that crystalline state of Form CSII does not change after grinding. The XRPD overlay of the solid before and after grinding is shown in FIG. 14.

### Example 13 Compressibility of Form CSII

ENERPAC manual tablet press was used for compression. 80 mg of Form CSII and Form A were weighed and added into the dies of a φ6 mm round tooling, compressed at 10 KN manually, then stored at room temperature for 24 h until complete elastic recovery. Hardness (H) was tested with an intelligent tablet hardness tester. Diameter (D) and thickness (L) were tested with caliper. Tensile strength of the powder was calculated with the following formula: T=2H/πDL*9.8. Under a certain force, the greater the tensile strength, the better the compressibility. The results are shown in Table 13. The results indicate that Form A is splintering during the tablet pressing process, Form CSII has better compressibility compared with Form A.

TABLE 13

| | | | | Tensile |
|---|---|---|---|---|
| | Thickness | Diameter | Hardness | strength |
| Form | (mm) | (mm) | (kgf) | (MPa) |
| Form A | N/A | N/A | N/A | N/A |
| Form CSII | 2.30 | 6.08 | 4.24 | 1.89 |

### Example 14 Adhesiveness of Form CSII

Approximately 30 mg of Form CSII and Form A were weighed and then added into the dies of φ8 mm round tooling, compressed at 10 kN and held for 30 s. The material sticking to the punch was recorded. The compression was repeated twice and the cumulative amount, maximum amount and average amount of material sticking to the punch during the compression were recorded. The results are shown in Table 14. The results indicate that the adhesion quantity of Form A is 3.2 times that of Form CSII and the adhesiveness of Form CSII is superior to that of Form A.

TABLE 14

| | Maximum | Average |
|---|---|---|
| Form | amount (mg) | amount (mg) |
| Form A | 0.260 | 0.190 |
| Form CSII | 0.06 | 0.06 |

The examples described above are only for illustrating the technical concepts and features of the present disclosure and intended to make those skilled in the art being able to understand the present disclosure and thereby implement it and should not be concluded to limit the protective scope of this disclosure. Any equivalent variations or modifications according to the spirit of the present disclosure should be covered by the protective scope of the present disclosure.

1. A crystalline form CSI of Compound I, wherein the X-ray powder diffraction pattern comprises characteristic peaks at 2theta values of 3.2°±0.2°, 5.6°±0.2° and 8.6°±0.2° using CuKα radiation,

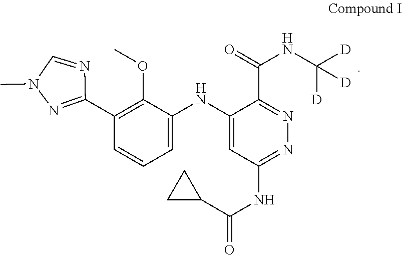 [SMILES: [2H]C([2H])([2H])NC(=O)c1nnc(NC(=O)C2CC2)cc1Nc1cccc(-c2ncn(C)n2)c1OC]

2. The crystalline form CSI of Compound I according to claim 1, wherein the X-ray powder diffraction pattern comprises one or two or three characteristic peaks at 2theta values of 11.8°±0.2°, 14.2°±0.2° and 15.0°±0.2° using CuKα radiation.

3. The crystalline form CSI of Compound I according to claim 1, wherein the X-ray powder diffraction pattern comprises one or two characteristic peaks at 2theta values of 17.3°±0.2° and 18.2°±0.2° using CuKα radiation.

4. The crystalline form CSI of Compound I according to claim 1, wherein, the X-ray powder diffraction pattern of form CSI is substantially as depicted in FIG. 1.

5. A process for preparing crystalline form CSI of Compound I according to claim 1, wherein the process comprises:
adding Compound I solid in a solvent,
stirring,
separating and
drying to obtain crystalline form CSI;
wherein the solvent is selected from
water,
a mixture of water and an alcohol,
a mixture of water and a ketone,
a mixture of water and a nitrile, or
a mixture of water and an ether.

6. The process according to claim 5, wherein said alcohol is a C1-C8 alcohol, said ketone is a C3-C6 ketone, said nitrile is a C2-C4 nitrile, said ether is a C2-C7 ether.

7. The process according to claim 5, wherein said alcohol is ethanol, said ketone is acetone, said nitrile is acetonitrile, said ether is 1,4-dioxane.

8-10. (canceled)

11. A pharmaceutical composition, said pharmaceutical composition comprises a therapeutically effective amount of the crystalline form CSI of Compound I according to claim 1 and pharmaceutically acceptable excipients.

12. A method of inhibiting TYK2 receptor, comprising administering to a subject in need thereof a therapeutically effective amount of the crystalline form CSI of Compound I according to claim 1.

13. A method for treating psoriasis, systemic lupus erythematosus, and Crohn's disease, comprising administering to a subject in need thereof a therapeutically effective amount of the crystalline form CSI of Compound I according to claim 1.
